SEQUENCE LISTING (2 sequences)

SEQ ID NO: 1 - protein, 181 aa, Homo sapiens
HPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPGLPPALPEPPGILAPQPPDVGSSDPLSMVGPSQGRSPSYAS

SEQ ID NO: 2 - DNA, 543 bp, Homo sapiens
caccccatccctgactccagtcctctcctgcaattcgggggccaagtccggcagcggtacctctacacagatgatgcccagcagacagaagcccacctggagatcagggaggatgggacggtggggggcgctgctgaccagagccccgaaagtctcctgcagctgaaagccttgaagccgggagttattcaaatcttgggagtcaagacatccaggttcctgtgccagcggccagatggggccctgtatggatcgctccactttgaccctgaggcctgcagcttccgggagctgcttcttgaggacggatacaatgtttaccagtccgaagcccacggcctcccgctgcacctgccagggaacaagtccccacaccgggaccctgcaccccgaggaccagctcgcttcctgccactaccaggcctgccccccgcactcccggagccacccggaatcctggccccccagccccccgatgtgggctcctcggaccctctgagcatggtgggaccttcccagggccgaagccccagctacgcttcc

(19) United States
(12) Patent Application Publication
(10) Pub. No.: US 2007/0265200 A1
(43) Pub. Date: Nov. 15, 2007

(54) Glycol Linked Fgf-21 Compounds

(75) Inventors: Wolfgang Glaesner, Indianapolis, IN (US); Radhakrishnan Rathnachalam, Carmel, IN (US); Rohn Lee Millican, Indianapolis, IN (US); Sheng-Hung Rainbow Tschang, Carmel, IN (US)

Correspondence Address: ELI LILLY & COMPANY, PATENT DIVISION, P.O. BOX 6288, INDIANAPOLIS, IN, 46206-6288, US

(73) Assignee: Eli Lilly and Company, Indianapolis, IN (US)

(21) Appl. No.: 10/592,016

(22) PCT Filed: Mar. 4, 2005
(86) PCT No.: PCT/US05/06799

§ 371 Date: Sep. 7, 2006

Related U.S. Application Data
(60) Provisional application No. 60/553,765, filed on Mar. 17, 2004.

(51) Int. Cl. A61K 38/18; A61P 3/00; A61P 3/04; A61P 3/10; C07K 14/50

(52) U.S. Cl. 514/12; 530/399

(57) ABSTRACT

The invention provides FGF-21 compounds covalently attached to at least one polyethylene glycol molecule or derivative thereof, resulting in a biologically active polypeptide with an extended elimination half-life and a slower clearance when compared to that of non-PEGylated polypeptide. These PEGylated FGF-21 compounds and compositions are useful in treating diabetes, obesity, and metabolic syndrome.

### FIELD OF THE INVENTION

The present invention relates to fibroblast growth factor 21 compounds covalently attached to one or more molecules of polyethylene glycol and methods useful in treating type 2 diabetes, obesity and metabolic syndrome.

### BACKGROUND OF THE INVENTION

Fibroblast growth factors are large polypeptides widely expressed in developing and adult tissues (Baird et al., Cancer Cells, 3:239-243, 1991) and play crucial roles in multiple physiological functions including angiogenesis, mitogenesis, pattern formation, cellular differentiation, metabolic regulation and repair of tissue injury (McKeehan et al., Prog. Nucleic Acid Res. Mol. Biol. 59:135-176, 1998). According to the published literature, the FGF family now consists of twenty-two members (Reuss et al., Cell Tissue Res. 313:139-157 (2003)).

Fibroblast growth factor 21 (FGF-21) has been reported to be preferentially expressed in the liver (Nishimura et al., Biochimica et Biophysica Acta, 1492:203-206, (2000); WO01/36640; and WO01/18172) and described as a treatment for ischemic vascular disease, wound healing, and diseases associated with loss of pulmonary, bronchia or alveolar cell function and numerous other disorders. More recently, FGF-21 has been shown to stimulate glucose-uptake in mouse 3T3-L1 adipocytes in the presence and absence of insulin, and to decrease fed and fasting blood glucose, triglycerides, and glucagon levels in ob/ob and db/db mice and 8 week old ZDF rats in a dose-dependant manner, thus, providing the basis for the use of FGF-21 as a therapy for treating diabetes and obesity (WO03/011213). In addition, FGF-21 has been shown to be effective in reducing the mortality and morbidity of critically ill patients (WO03/059270).

The present invention is based on the finding that covalent attachment of one or more molecules of PEG to particular residues of an FGF-21 compound results in a biologically active, PEGylated FGF-21 compound with an extended elimination half-life and reduced clearance when compared to that of native FGF-21.

The PEGylated FGF-21 compounds of the invention have greater usefulness as a therapeutic as well as greater convenience of use than native FGF-21 because they retain all or a portion of the biological activity of native FGF-21 yet have an extended time action when compared to that of the native FGF-21.

Therefore, PEGylated FGF-21 compounds of the present invention are useful to treat subjects with disorders including, but not limited to, type 2 diabetes, obesity, and metabolic syndrome, with particular advantages being that the PEGylated FGF-21 compounds of the invention present the potential for increased efficacy due to constant exposure and require fewer doses, increasing both the convenience to a subject in need of such therapy and the likelihood of a subject's compliance with dosing requirements.

### SUMMARY OF THE INVENTION

The invention described herein provides FGF-21 compounds covalently attached to one or more molecules of polyethylene glycol (PEG), or a derivative thereof wherein each PEG is attached at a cysteine or lysine amino acid residue of the polypeptide, resulting in PEGylated FGF-21 compounds with an extended time action compared to a non-PEGylated FGF-21 compound.

An embodiment of the invention is a PEGylated FGF-21 compound comprising the amino acid sequence of FGF-21 as shown in SEQ ID NO: 1 wherein at least one PEG molecule is covalently attached at a cysteine residue substituted for the native residue at positions selected from the group consisting of D25C, D38C, L58C, K59C, P60C, K69C, D79C, H87C, E91C, E101C, D102C, L114C, L116C, K122C, R126C, P130C, P133C, or P140C.

Another embodiment of the invention is a PEGylated FGF-21 compound comprising the amino acid sequence as shown in SEQ ID NO: 1 covalently attached to a PEG molecule at one or two of the residues selected from the group consisting of lysine at position 56, 59, 69 and 122.

Yet another embodiment of the present invention encompasses pharmaceutical compositions of PEGylated FGF-21 compounds and methods of treating a patient suffering from type 2 diabetes, obesity, insulin resistance, hyperinsulinemia, glucose intolerance, hyperglycemia, or metabolic syndrome comprising administering to said patient a therapeutically effective amount of a PEGylated FGF-21 compound.

### DETAILED DESCRIPTION OF THE INVENTION

For purposes of the present invention, as disclosed and claimed herein, the following terms are as defined below.

FGF-21 is a 208 amino acid polypeptide containing a 27 amino acid leader sequence. Human FGF-21 is highly identical to mouse FGF-21 (˜79% amino acid identity) and rat FGF-21 (˜80% amino acid identity). Human FGF-21 is the preferred polypeptide of the present invention but it is recognized that one with skill in the art could readily use analogs, muteins, or derivatives of human FGF-21 or an alternative mammalian FGF-21 polypeptide sequence for the uses described herein.

The amino acid positions of the present invention are determined from the mature, wild type or native human 181 amino acid FGF-21 polypeptide as shown below (SEQ ID NO: 1):

```
1 10 20
His Pro Ile Pro Asp Ser Ser Pro Leu Leu Gln Phe Gly Gly Gln Val Arg Gln Arg Tyr
                                                                        30 40
Leu Tyr Thr Asp Asp Ala Gln Gln Thr Glu Ala His Leu Glu Ile Arg Glu Asp Gly Thr
                                                                        50 60
Val Gly Gly Ala Ala Asp Gln Ser Pro Glu Ser Leu Leu Gln Leu Lys Ala Leu Lys Pro
                                                                        70 80
Gly Val Ile Gln Ile Leu Gly Val Lys Thr Ser Arg Phe Leu Cys Gln Arg Pro Asp Gly
                                                                        90 100
Ala Leu Tyr Gly Ser Leu His Phe Asp Pro Glu Ala Cys Ser Phe Arg Glu Leu Leu Leu
                                                                        110 120
Gln Asp Gly Tyr Asn Val Tyr Gln Ser Glu Ala His Gly Leu Pro Leu His Leu Pro Gly
                                                                        130 140
Asn Lys Ser Pro His Arg Asp Pro Ala Pro Arg Gly Pro Ala Arg Phe Leu Pro Leu Pro
                                                                        150 160
Gly Leu Pro Pro Ala Leu Pro Gln Pro Pro Gly Ile Leu Ala Pro Gln Pro Pro Asp Val
                                                                        170 180
Gly Ser Ser Asp Pro Leu Ser Met Val Gly Pro Ser Gln Gly Arg Ser Pro Ser Tyr Ala
Ser
```

The corresponding DNA sequence coding for the mature human 181 amino acid FGF-21 polypeptide is (SEQ ID NO:2):

CACCCCATCCCTGACTCCAGTCCTCTCCTGCAATTCGGGGGCCAAGTCCG
GCAGCGGTACCTCTACACAGATGATGCCCAGCAGACAGAAGCCCACCTGG
AGATCAGGGAGGATGGGACGGTGGGGGGCGCTGCTGACCAGAGCCCCGAA
AGTCTCCTGCAGCTGAAAGCCTTGAAGCCGGGAGTTATTCAAATCTTGGG
AGTCAAGACATCCAGGTTCCTGTGCCAGCGGCCAGATGGGGCCCTGTATG
GATCGCTCCACTTTGACCCTGAGGCCTGCAGCTTCCGGGAGCTGCTTCTT
GAGGACGGATACAATGTTTACCAGTCCGAAGCCCACGGCCTCCCGCTGCA
CCTGCCAGGGAACAAGTCCCCACACCGGGACCCTGCACCCCGAGGACCAG
CTCGCTTCCTGCCACTACCAGGCCTGCCCCCCGCACTCCCGGAGCCACCC
GGAATCCTGGCCCCCCAGCCCCCCGATGTGGGCTCCTCGGACCCTCTGAG
CATGGTGGGACCTTCCCAGGGCCGAAGCCCCAGCTACGCTTCC

The FGF-21 useful in the methods of the present invention is preferably human FGF-21 as shown in SEQ ID NO: 1, analogs, muteins, and derivatives thereof, hereinafter collectively known as FGF-21 compounds. FGF-21 compounds have sufficient homology to FGF-21 such that the compounds have the ability to bind to the FGF-21 receptor and initiate a signal transduction pathway resulting in glucose uptake stimulation or other physiological effects as described herein. For example, FGF-21 compounds can be tested for glucose uptake activity using a cell-based assay such as that described in Example 1.

The term “PEGylated” when referring to a FGF-21 compound of the present invention refers to a FGF-21 compound that is chemically modified by covalent attachment of one or more molecules of polyethylene glycol or a derivative thereof. Furthermore, it is intended that the term “PEG” refers to polyethylene glycol or a derivative thereof as are known in the art (see, e.g., U.S. Pat. Nos.: 5,900,461; 5,932,462; 6,436,386; 6,448,369; 6,437,025; 6,448,369; 6,495,659; 6,515,100 and 6,514,491). Optionally, the PEG molecules may be attached to the FGF-21 compound via a linker or spacer molecule (see exemplary spacer molecules described in U.S. Pat. No. 6,268,343).

A “subject” or “patient” is a mammal, preferably a human.

Type 2 diabetes is characterized by excess glucose production in spite of the availability of insulin, and circulating glucose levels remain excessively high as a result of inadequate glucose clearance.

Glucose intolerance can be defined as an exceptional sensitivity to glucose.

Hyperglycemia is defined as an excess of sugar (glucose) in the blood.

Hypoglycemia, also called low blood sugar, occurs when your blood glucose level drops too low to provide enough energy for your body's activities.

Hyperinsulinemia is defined as a higher-than-normal level of insulin in the blood.

Insulin resistance is defined as a state in which a normal amount of insulin produces a subnormal biologic response.

Metabolic syndrome can be defined as a cluster of at least three of the following signs: abdominal fat—in most men, a 40-inch waist or greater; high blood sugar—at least 110 milligrams per deciliter (mg/dl) after fasting; high triglycerides—at least 150 mg/dL in the bloodstream; low HDL—less than 40 mg/dl; and, blood pressure of 130/85 or higher.

Native or wild type refers to the mature human 181 amino acid FGF-21 polypeptide as shown in SEQ ID NO:1.

The term “amino acid” is used herein in its broadest sense, and includes naturally occurring amino acids as well as non-naturally occurring amino acids, including amino acid variants and derivatives. One skilled in the art will recognize, in view of this broad definition, that reference herein to an amino acid includes, for example, naturally occurring proteogenic L-amino acids; D-amino acids; chemically modified amino acids such as amino acid variants and derivatives; naturally occurring non-proteogenic amino acids such as norleucine, β-alanine, ornithine, etc.; and chemically synthesized compounds having properties known in the art to be characteristic of amino acids. Examples of non-naturally occurring amino acids include α-methyl amino acids (e.g., α-methyl alanine), D-amino acids, histidine-like amino acids (e.g., 2-amino-histidine, β-hydroxy-histidine, homohistidine, α-fluoromethyl-histidine and α-methyl-histidine), amino acids having an extra methylene in the side chain (“homo” amino acids) and amino acids in which a carboxylic acid functional group in the side chain is replaced with a sulfonic acid group (e.g., cysteic acid). Preferably, however, the FGF-21 compounds of the present invention comprise only naturally occurring amino acids except as otherwise specifically provided herein.

In the nomenclature used herein to designate FGF-21 compounds, amino acids are identified using the three-letter code or alternatively using the standard one letter code. Mutations are designated by the three-letter code for the original amino acid, followed by the amino acid number, followed by the three-letter code for the replacement amino acid. The numerical designations of each mutein is based on the 181 amino acid sequence of mature, wild-type, human FGF-21. For example, a substitution for lysine at position 59 (i.e. Lys59) with cysteine (Cys) is designated as Lys59Cys or K59C. In a similar fashion, the double substitution for isoleucine at position 152 and serine at position 163 (Ile152, Ser163) with the negatively charged amino acid, glutamate (Glu) is designated as Ile152Glu/Ser163Glu or 1152E/S163E.

The term “native” or “wild type” refers to a polypeptide that has an amino acid sequence that is identical to one found in nature. The term “native” or “wild type” is intended to encompass allelic variants of the polypeptide in question.

“In vitro potency” as used herein, is the measure of glucose uptake of a pegylated-FGF-21 compound in a cell-based assay and is a measure of the biological potency of the FGF-21 compound. In vitro potency is expressed as the “EC₅₀” which is the effective concentration of compound that results in 50% activity in a single dose-response experiment. For the purposes of the present invention, in vitro potency is determined using a glucose uptake assay that employs 3T3-L1 cells (Example 1).

The term “plasma half-life” refers to the time in which half of the relevant molecules circulate in the plasma prior to being cleared. An alternatively used term is “elimination half-life.” The terms “extended time action” or “longer time action” used in the context of plasma half-life or elimination half-life indicates there is a statistically significant increase in the half-life of a PEGylated FGF-21 compound relative to that of the reference molecule (e.g., the non-PEGylated form of the polypeptide or the native polypeptide) as determined under comparable conditions. Preferably a PEGylated FGF-21 compound of the present invention has an elimination half-life greater than that of a comparable non-PEGylated FGF-21 compound. The half-life reported herein in Example 5 is the elimination half-life; it is that which corresponds to the terminal log-linear rate of elimination. Those of skill in the art appreciate that half-life is a derived parameter that changes as a function of both clearance and volume of distribution.

Clearance is the measure of the body's ability to eliminate a drug. As clearance decreases due, for example, to modifications to a drug, half-life would be expected to increase. However, this reciprocal relationship is exact only when there is no change in the volume of distribution. A useful approximate relationship between the terminal log-linear half-life (t_{1/2}), clearance (C), and volume of distribution (V) is given by the equation: t_{1/2}≈0.693 (V/C). Clearance does not indicate how much drug is being removed but, rather, the volume of biological fluid such as blood or plasma that would have to be completely freed of drug to account for the elimination. Clearance is expressed as a volume per unit of time (See Example 5).

The present invention describes modifications to FGF-21 compounds that result in extended elimination half-life and/or reduced clearance. Incorporation of 1 or 2 Cys residues into particular amino acid sites of the peptide provides a thiol group to which a polyethylene glycol (PEG) or PEG derivative may be covalently attached resulting in a PEGylated FGF-21 compound. Additionally, the lysine residues of the analogs or fragments of the invention may be covalently attached to one or more molecules of PEG or a PEG derivative resulting in a molecule with extended elimination half-life and/or reduced clearance.

A human FGF-21 mutein is defined as comprising human FGF-21 in which at least one amino acid of the wild-type mature protein has been substituted by another amino acid. Examples of FGF-21 muteins are described in U.S. patent application 60/528,582 herein incorporated by reference. Generally speaking, a mutein possesses some modified property, structural or functional, of the wild-type protein. For example, the mutein may have enhanced or improved physical stability in concentrated solutions (e.g., less hydrophobic mediated aggregation), while maintaining a favorable bioactivity profile. The mutein may possess increased compatibility with pharmaceutical preservatives (e.g., m-cresol, phenol, benzyl alcohol), thus enabling the preparation of a preserved pharmaceutical formulation that maintains the physiochemical properties and biological activity of the protein during storage. Accordingly, muteins with enhanced pharmaceutical stability when compared to wild-type FGF-21, have improved physical stability in concentrated solutions under both physiological and preserved pharmaceutical formulation conditions, while maintaining biological potency. As used herein, these terms are not limiting, it being entirely possible that a given mutein has one or more modified properties of the wild-type protein.

Accordingly, the present invention provides the pegylation of muteins of FGF-21, or a biologically active peptide thereof at a lysine residue or a cysteine residue. Examples of FGF-21 muteins with enhanced pharmaceutical stability include the substitution with a charged and/or polar but uncharged amino acid for one or more of the following: glycine 42, glutamine 54, arginine 77, alanine 81, leucine 86, phenylalanine 88, lysine 122, histidine 125, arginine 126, proline 130, arginine 131, leucine 139, alanine 145, leucine 146, isoleucine 152, alanine 154, glutamine 156, glycine 161, serine 163, glycine 170, or serine 172 wherein the numbering of the amino acids is based on SEQ ID NO:1.

Additional muteins of FGF-21 muteins with enhanced pharmaceutical stability include FGF-21 with the substitution of a cysteine for two or more of the following: arginine 19, tyrosine 20, leucine 21, tyrosine 22, threonine 23, aspartate 24, aspartate 25, alanine 26, glutamine 27, lutamine 28, alanine 31, leucine 33, isoleucine 35, leucine 37, valine 41, glycine 42, glycine 43, glutamate 50, glutamine 54, leucine 58, valine 62, leucine 66, glycine 67, lysine 69, arginine 72, phenylalanine 73, glutamine 76, arginine 77, aspartate 79, glycine 80, alanine 81, leucine 82, glycine 84, serine 85, proline 90, alanine 92, serine 94, phenylalanine 95, leucine 100, aspartate 102, tyrosine 104, tyrosine 107, serine 109, glutamate 110, proline 115, histidine 117, leucine 118, proline 119, asparagine 121, lysine 122, serine 123, proline 124, histidine 125, arginine 126, aspartate 127, alanine 129, proline 130, glycine 132, alanine 134, arginine 135, leucine 137, proline 138, or leucine 139, wherein the numbering of the amino acids is based on SEQ ID NO:1.

Specific muteins of FGF-21 with engineered disulfide bonds, in addition to the naturally occurring one at Cys75-Cys93, are as follows: Gln76Cys-Ser109Cys, Cys75-Ser85Cys, Cys75-Ala92Cys, Phe73Cys-Cys93, Ser123Cys-His125-Cys, Asp102Cys-Tyr104Cys, Asp127Cys-Gly132Cys, Ser94Cys-Glu110Cys, Pro115Cys-His117Cys, Asn121Cys-Asp127Cys, Leu100Cys-Asp102Cys, Phe95Cys-Tyr107Cys, Arg19Cys-Pro138Cys, Tyr20Cys-Leu139Cys, Tyr22Cys-Leu137Cys, Arg77Cys-Asp79Cys, Pro90Cys-Ala92Cys, Glu50Cys-Lys69Cys, Thr23Cys-Asp25Cys, Ala31Cys-Gly43Cys, Gln28Cys-Gly43Cys, Thr23Cys-Gln28Cys, Val41Cys-Leu82Cys, Leu58Cys-Val62Cys, Gln54Cys-Leu66Cys, Ile35Cys-Gly67Cys, Gly67Cys-Arg72Cys, Ile35Cys-Gly84Cys, Arg72Cys-Gly84Cys, or Arg77Cys-Ala81Cys, wherein the numbering of the amino acids is based on SEQ ID NO:1. Preferred muteins with engineered disulfide bonds are Tyr22Cys-Leu139Cys; Asp24Cys-Arg135Cys; Leu118Cys-Gly132Cys; His117Cys-Pro130Cys; His117Cys-Ala129Cys; Leu82Cys-Pro119Cys; Gly80Cys-Ala129Cys; Gly43Cys-Pro124Cys; Gly42Cys-Arg126Cys; Gly42Cys-Pro124Cys; Gln28Cys-Pro124Cys; Gln27Cys-Ser123Cys; Ala26Cys-Lys122Cys; or Asp25Cys-Lys122Cys. Most preferred muteins with engineered disulfide bonds are Leu118Cys-Ala134Cys; Leu21Cys-Leu33Cys; Ala26Cys-Lys122Cys; Leu21Cys-Leu33Cys/Leu118Cys-Ala134Cys. For the purpose of the present invention, when pegylating muteins with engineered disulfide bonds, a cysteine residue may be substituted and pegylated at only one additional position at any given time, since substituting two or more positions with a cysteine may result in an intrachain disulfide bond that would preclude the ability to pegylate the polypeptide at that position.

The family of FGF proteins have a common β-trefoil or β-sheet structure as identified by crystallography (Harmer et al., Biochemistry 43:629-640 (2004)). An ordinary skilled artisan recognizes that such analysis of FGF-21 enables the determination of which amino acid residues are surface exposed compared to amino acid residues that are buried within the tertiary structure of the protein. Therefore, it is an embodiment of the present invention to substitute a cysteine residue only for an amino acid residue that is a surface exposed residue. The location of an amino acid residue being replaced with a cysteine is determined by homology modeling utilizing Accelrys software (Incyte). By this method, each residue is mutated to cysteine, the energy minimized and a calculation is performed to determine the accessibility of the residue for different solvent radii. Typically 1.4↑ to 7.0Å are the solvent radii used (1.4Å is the approximate radius of a water molecule). It is preferable that cysteine substitutions determined by the above homology method be incorporated at one or more amino acid residues at positions arginine 19, leucine 21, alanine 26, glutamine 28, threonine 29, glutamate 30, arginine 36, glycine 39, glycine 42, glutamate 50, lysine 56, glycine 61, glutamine 64, isoleucine 65, valine 68, threonine 70, serine 71, arginine 77, alanine 81, serine 85, leucine 86, proline 90, alanine 92, serine 94, leucine 98, tyrosine 107, glutamine 108, histidine 112, glycine 113, serine 123, or proline 124. More preferably, cysteine substitutions may be incorporated at positions aspartate 24, glutamine 27, glutamate 37, threonine 40, alanine 44, aspartate 46, proline 49, alanine 57, phenylalanine 88, aspartate 89, valine 106, glutamae 110, alanine 111, proline 115, glycine 120, or leucine 139. Even more preferably, cysteine substitutions may be incorporated at positions glutamine 18, alanine 45, glutamine 47, serine 48, proline 78, tyrosine 83, leucine 99, glycine 103, histidine 125, proline 128, arginine 131, glycine 132, or proline 138. Most preferably, cysteine substitutions may be incorporated at positions aspartate 25, aspartate 38, leucine 58, lysine 59, proline 60, lysine 69, aspartate 79, histidine 87, glutamate 91, glutamate 101, aspartate 102, leucine 114, leucine 116, lysine 122, arginine 126, proline 130, proline 133, orproline 140. The resulting FGF-21 compound may be PEGylated at the substituted Cys amino acid resulting in a modified molecule that retains all or a portion of a biological activity while having a longer half-life than that of the unmodified compound or than that of a native compound.

Alternatively, in the invention provides FGF-21 compounds PEGylated at one, two or three of the lysine residues at positions 56, 59, 69 and 122. The resulting molecule may be PEGylated at the lysine amino acids resulting in a modified molecule that retains all or a portion of a biological activity while having an extended time action when compared to that of the unmodified molecule or a native molecule.

An FGF-21 compound also includes an “FGF-21 derivative” which is defined as a molecule having the amino acid sequence of FGF-21 or an FGF-21 analog, but additionally having a chemical modification of one or more of its amino acid side groups, α-carbon atoms, terminal amino group, or terminal carboxylic acid group. A chemical modification includes, but is not limited to, adding chemical moieties, creating new bonds, and removing chemical moieties.

Modifications at amino acid side groups include, without limitation, acylation of lysine ε-amino groups, N-alkylation of arginine, histidine, or lysine, alkylation of glutamic or aspartic carboxylic acid groups, and deamidation of glutamine or asparagine. Modifications of the terminal amino group include, without limitation, the des-amino, N-lower alkyl, N-di-lower alkyl, and N-acyl modifications. Modifications of the terminal carboxy group include, without limitation, the amide, lower alkyl amide, dialkyl amide, and lower alkyl ester modifications. Furthermore, one or more side groups, or terminal groups, may be protected by protective groups known to the ordinarily-skilled protein chemist. The α-carbon of an amino acid may be mono- or dimethylated.

Once a polypeptide for use in the invention is prepared and purified, it is modified by covalently linking at least one PEG molecule to a Cys or Lys residue or to the amino-terminal amino acid. It is difficult to endow delicate polypeptide or protein molecules with suitable new properties by attaching polymers without causing loss of their functionality. A wide variety of methods have been described in the art to produce covalently conjugated to PEG and the specific method used for the present invention is not intended to be limiting (for review article see, Roberts, M. et al. Advanced Drug Delivery Reviews, 54:459-476, 2002). PEGylation of proteins may overcome many of the pharmacological and toxicological/immunological problems associated with using peptides or proteins as therapeutics. However, for any individual polypeptide it is uncertain whether the PEGylated form of the polypeptide will have significant loss in bioactivity as compared to the unPEGylated form of the polypeptide.

The bioactivity of PEGylated proteins can be effected by factors such as: i) the size of the PEG molecule; ii) the particular sites of attachment; iii) the degree of modification; iv) adverse coupling conditions; v) whether a linker is used for attachment or whether the polymer is directly attached; vi) generation of harmful co-products; vii) damage inflicted by the activated polymer; or viii) retention of charge. Depending on the coupling reaction used, polymer modification of cytokines, in particular, has resulted in dramatic reductions in bioactivity. [Francis, G. E., et al., (1998) PEGylation of cytokines and other therapeutic proteins and peptides: the importance of biological optimization of coupling techniques, Intl. J. Hem. 68:1-18].

PEGylated FGF-21 compounds of the present invention have an in vitro biological activity that is comparable or less than that of native FGF-21. Although some PEGylated FGF-21 compounds of the invention may have biological activity lower than that of native FGF-21 as measured in a particular assay, this activity decrease is compensated by the compound's extended half-life and/or lower clearance value and may even be a favorable characteristic for an FGF-21 compound with an extended elimination half-life.

In its typical form most useful for polypeptide modification, PEG is a linear polymer with terminal hydroxyl groups and has the formula: CH₃O—(CH₂CH₂O)n-CH₂CH₂—OH, where n is from about 8 to about 4000. The terminal hydrogen may be substituted with a protective group such as an alkyl or alkanol group. Preferably, PEG has at least one hydroxy group, more preferably it is a terminal hydroxy group. It is this hydroxy group which is preferably activated to react with the polypeptide. There are many forms of PEG useful for the present invention. Numerous derivatives of PEG exist in the art and are suitable for use in the invention (Zalipsky, S. Bioconjugate Chem. 6:150-165, 1995). The PEG molecule covalently attached to FGF-21 compounds in the present invention is not intended to be limited to a particular type. PEG's molecular weight is preferably from 500-100,000 daltons, more preferably 10.000-80,000 daltons, even more preferably from 20,000-60,000 daltons and most preferably from 20,000-40,000 daltons. PEG may be linear or branched and PEGylated FGF-21 compounds of the invention may have 1, 2, 3, 4, 5 or 6 PEG molecules attached to the peptide. It is most preferably that there be one PEG molecule per PEGylated FGF-21 compound molecule; however, when there are more than PEG molecules per peptide molecule, it is preferred that there be no more than six.

The present invention provides FGF-21 compounds with one or more PEG molecules covalently attached thereto. PEG derivatives such as PEG-maleimide, vinylsulfone, iodoacetamide, and orthopyridyl disulfide have been developed for PEGylation on cysteine residues (Goodson et al., Biotechnology 8:343-346 (1990); Kogan et al., Synth. Commun. 22: 2417-2424 (1992); Morpurgo et al., Bioconjug. Chem. 7:363-368 (1996); and Woghiren et al., Bioconjug. Chem. 4:314-318 (1993)). The preferred method for preparing the PEGylated FGF-21 compounds of the present invention involves the use of PEG-maleimide to directly attach PEG to a thiol group of the peptide. The introduction of a thiol functionality can be achieved by adding or inserting a Cys residue onto or into the polypeptide at positions described above. A thiol functionality can also be introduced onto the side-chain of the peptide (e.g. acylation of lysine ε-amino group of a thiol-containing acid). A PEGylation process of the present invention utilizes Michael addition to form a stable thioether linker. The reaction is highly specific and takes place under mild conditions in the presence of other functional groups. PEG maleimide has been used as a reactive polymer for preparing well-defined, bioactive PEG-protein conjugates. It is preferable that the procedure uses a molar excess of a thiol-containing FGF-21 compound relative to PEG maleimide to drive the reaction to completion. The reactions are preferably performed between pH 4.0 and 9.0 at room temperature for 15 to 40 hours. The excess of unPEGylated thiol-containing peptide is readily separated from the PEGylated product by conventional separation methods. Exemplary conditions required for PEGylation of FGF-21 compounds are set forth in Examples 2 and 3. Cysteine PEGylation may be performed using PEG maleimide or bifurcated PEG maleimide.

The FGF-21 compounds of the present invention may be generated and/or isolated by any means known in the art such as described in Sambrook et al., Molecular Cloning: A Laboratory Manual, Cold Spring Harbor Laboratory Press, NY (1989).

Various methods of protein purification may be employed and such methods are known in the art and described, for example, in Deutscher, Methods in Enzymology 182: 83-9 (1990) and Scopes, Protein Purification: Principles and Practice, Springer-Verlag, NY (1982). The purification step(s) selected will depend, for example, on the nature of the production process used for FGF-21

FGF-21 compounds have a variety of biological activities. FGF-21 is particularly promising as a treatment for non-insulin dependent diabetes mellitus (NIDDM, type 2) as it does not present a risk of hypoglycemia as do present NIDDM treatments. FGF-21 is also contemplated to be a treatment for obesity and metabolic syndrome.

It is contemplated that a use of a PEGylated FGF-21 compounds of the present invention includes use in the manufacture of a medicament for the treatment of type 2 diabetes, obesity and metabolic syndrome. PEGylation of a FGF-21 compound may be combined with other modifications known in the art to increase FGF-21 half-life and thereby increase the half-life of the compound even further than PEGylation alone or the other modification method alone.

As used herein, the term “FGF-21 compound” also includes pharmaceutically acceptable salts of the compounds described herein. An FGF-21 compound of this invention can possess a sufficiently acidic, a sufficiently basic, or both functional groups, and accordingly react with any of a number of inorganic bases, and inorganic and organic acids, to form a salt.

The PEGylated FGF-21 compounds of the present invention are particularly suited for parenteral administration, they can be also be delivered orally, by nasal administration, or by inhalation. Parenteral administration can include, for example, systemic administration, such as by intramuscular, intravenous, subcutaneous, or intraperitoneal injection. The PEGylated FGF-21 compounds can be administered to the subject in conjunction with an acceptable pharmaceutical carrier, diluent or excipient as part of a pharmaceutical composition for treating the diseases discussed above. The pharmaceutical composition can be a solution or, if administered parenterally, a suspension of the FGF-21. Suitable pharmaceutical carriers may contain inert ingredients which do not interact with the peptide or peptide derivative. Standard pharmaceutical formulation techniques may be employed such as those described in Remington's Pharmaceutical Sciences, Mack Publishing Company, Easton, Pa. Suitable pharmaceutical carriers for parenteral administration include, for example, sterile water, physiological saline, bacteriostatic saline (saline containing about 0.9% mg/ml benzyl alcohol), phosphate-buffered saline, Hank's solution, Ringer's-lactate and the like. Some examples of suitable excipients include lactose, dextrose, sucrose, trehalose, sorbitol, and mannitol.

The PEGylated FGF-21 compounds of the invention may be formulated for administration such that blood plasma levels are maintained in the efficacious range for extended time periods.

A “therapeutically effective amount” of a PEGylated FGF-21 compound is the quantity that results in a desired therapeutic and/or prophylactic effect without causing unacceptable side-effects when administered to a subject. A “desired therapeutic effect” includes one or more of the following: 1) an amelioration of the symptom(s) associated with the disease or condition; 2) a delay in the onset of symptoms associated with the disease or condition; 3) increased longevity compared with the absence of the treatment; and 4) greater quality of life compared with the absence of the treatment. For example, an “effective amount” of a PEGylated FGF-21 compound for the treatment of type 2 diabetes is the quantity that would result in greater control of blood glucose concentration than in the absence of treatment, thereby resulting in a delay in the onset of diabetic complications such as retinopathy, neuropathy or kidney disease. An “effective amount” of a PEGylated FGF-21 compound for the prevention of diabetes is the quantity that would delay, compared with the absence of treatment, the onset of elevated blood glucose levels that require treatment with anti-hypoglycaemic drugs such as sulfonyl ureas, thiazolidinediones, insulin and/or bisguanidines. Moreover, a “therapeutically effective amount” of the PEGylated FGF-21 compound administered to a subject will also depend on the type and severity of the disease and on the characteristics of the subject, such as general health, age, sex, body weight and tolerance to drugs.

Those skilled in the art can readily optimize pharmaceutically effective dosages and administration regimens for therapeutic compositions comprising a PEGylated FGF-21 compound, as determined by good medical practice and the clinical condition of the individual patient. A typical dose range for the PEGylated FGF-21 compounds of the present invention will range from about 0.01 mg per day to about 1000 mg per day for an adult. Preferably, the dosage ranges from about 0.1 mg per day to about 100 mg per day, more preferably from about 1.0 mg/day to about 10 mg/day. Most preferably, the dosage is about 1-5 mg/day. The appropriate dose of a PEGylated FGF-21 compound administered will result in lowering blood glucose levels and increasing energy expenditure by faster and more efficient glucose utilization, and thus is useful for treating type 2 diabetes, obesity and metabolic syndrome.

Having now described the present invention in detail, the same will be more clearly understood by reference to the following examples, which are included herewith for purposes of illustration only and are not intended to be limiting of the invention.

All patents and publications referred to herein are expressly incorporated by reference.

### Preparation 1

### Expression and Purification of an FGF-21 Compound in E. coli

The bacterial expression vector pET30a is used for bacterial expression in this example. (Novagen, Inc., Madison, Wis.)). pET30a encodes kanamycin antibiotic resistance gene and contains a bacterial origin of replication (“ori”), a strong T7 phage-IPTG inducible promoter, a ribosome binding site (“RBS”), and suitable MCS with a number of unique restriction endonuclease cleavage sites. Conveniently for purification purpose, the vector can encode His- and S-tags for N-terminal peptide fusions, as well as, a C-terminal His-tag fusion. However, for purposes of the present invention, the cDNA encoding an FGF-21 compound is inserted between restriction sites NdeI and BamHI, respectively, and the resulting construct does not take advantage of either of the described tags.

The nucleic acid sequence encoding an FGF-21 compound, lacking the leader sequence but substituted with a methionine residue, is amplified from a cDNA clone using PCR oligonucleotide primers, which anneal to the 5′ and 3′ ends of the open reading frame. Additional nucleotides, containing recognition sites for restriction enzymes NdeI and BamHI, are added to the 5′ and 3′ sequences, respectively.

For cloning, the 5′ forward and 3′ reverse PCR primers have nucleotides corresponding or complementary to a portion of the coding sequence of an FGF-21 compound-encoding nucleic acid according to methods known in the art. One of ordinary skill in the art would appreciate that the point in a polynucleotide sequence where primers begin can be varied.

The amplified nucleic acid fragments and the vector pET30a are digested with NdeI and BamHI restriction enzymes and the purified digested DNA fragments are then ligated together. Insertion of an FGF-21 compound-encoding DNA into the restricted pET30a vector places the FGF-21 compound polypeptide coding region including its associated stop codon downstream from the IPTG-inducible promoter and in-frame with an initiating ATG codon. The associated stop codon, TAG, prevents translation of the six-histidine codons downstream of the insertion point.

The ligation mixture is transformed into competent E. coli cells using standard procedures such as those described in Current Protocols in Molecular Biology (John Wiley & Sons, Inc.).

Transformation reactions are plated on LB/Kanamycin plates and after an overnight growth transformants are picked for plasmid preparations or lysed in situ for screening by PCR. Positive recombinant plasmids, containing desired FGF-21 compound inserts, are identified by restriction analysis followed by DNA sequence analysis. Those plasmids are subsequently used to transform expression strains for protein production.

E. coli strains BL21(DE3), BL21(DE3)STAR or BL21(DE3) RP, are used for expressing an FGF-21 compound. These strains, which are only some of many that are suitable for expressing an FGF-21 compound, are available commercially from Novagen, Inc., Invitrogen and Stratagen, respectively. Transformants are identified by their ability to grow on LB plates in the presence of kanamycin.

Clones containing the desired constructs are grown overnight (o/n) in liquid culture in LB media supplemented with kanamycin (30 μg/ml). The o/n culture is used to inoculate a large culture, at a dilution of approximately 1:25 to 1:250. The cells are grown to an optical density of 0.6 (“OD600”) at 600 nm. Isopropyl-b-D-thiogalactopyranoside (“IPTG”) is then added to a final concentration of 1 mM to induce transcription from the lac repressor sensitive promoter, by inactivating the lacI repressor. Cells subsequently are incubated further for 3 to 12 hours. Cells are then harvested by centrifugation, pellets washed with 50 mM Tris buffer, pH 8.0 and stored at −20° C. until purification. FGF-21 is expressed in the insoluble fraction i.e inclusion bodies (or granules) of E. coli. The expression level typically observed for an FGF-21 compound is 50 mg/L. The subsequent purification process starts with solubilization of the granules and refolding of the variants followed by four chromatographic steps.

To purify an FGF-21 compound from E coli, the granules are solubilzed in 50 mM Tris, pH 9.0, 7M Urea and 1 mM DTT through a pH ramp to pH 11.0, at room temperature for 1 hour with stirring. The protein is then captured on a Q-Sepharose column using the same buffer described above, and eluted with a linear gradient of 0-400 mM NaCl. The Q-Sepharose pool is then treated with 10 mM DTT, for two hours, at RT, to reduce all disulfide bonds. The pool is then diluted 10-fold so that the buffer concentration is as follows: 50 mM Tris, pH 9.0, 7 M Urea, 10 mM Cysteine, 1 mM DTT with a protein concentration of approximately 250-500 μg/ml. After another two-hour incubation under reducing conditions at RT, to obtain the protein in a free disulfide form, the pool is then dialyzed into 20 mM glycine, pH 9.0 for approximately 48 hours so that the correct disulfide bonds can be formed.

Reversed-phase HPLC chromatography, on a Vydac C18 column and 0.1% TFA/0-50% CH₃CN as a mobile phase is used as an initial purification step. This column is used to concentrate an FGF-21 compound and removes contaminating endotoxin.

The next purification step is size exclusion chromatography on a Superdex 35/600 column performed in 1×PBS buffer, pH7.4. At this step an FGF-21 compound is ˜95% pure. The last step involves MonoQ chromatography in 50 mM Tris, pH 8.0 and elution with a linear gradient of 0-300 mM NaCl, which usually yields >97% pure protein.

### Preparation 2

### Expression and Purification an FGF-21 compound in HEK293EBNA Cells

Alternatively, FGF-21 compounds are produced in a mammalian cell expression system using HEK293EBNA cells (EdgeBiosystems, Gaiethersburg, Md.). FGF-21 compounds are subcloned in the proprietary expression vector representing a modification of commercially available pEAK10, between NheI and XbaI restriction sites in the MCS. The cDNA sequence encoding an FGF-21 compound is fused in frame with the Igκ leader sequence to enhance secretion of the desired product in the tissue culture media. The expression is driven by the strong viral CMV promoter. HEK293EBNA cells are transiently transfected using a standard transfection reagent such as Fugene (Roche Diagnostics, Indianapolis Ind., USA) and the appropriate amount of recombinant plasmid, either as a monolayer or suspension culture, at the adequate cell density. Cells are incubated at 37° C. and 5% C0₂, in serum free media, and collections are made every day for 5 days. Typically the expression level in the HEK293EBNA suspension culture is ˜30 mg/L. The expression of an FGF-21 compound in mammalian cells yields the natural N-terminal sequence, HPIP, i.e. without a methionine residue at the N-terminus.

To purify an FGF-21 compound from HEK293EBNA cells, concentrated cell culture supernatant loaded onto a 10 ml Fast Flow Q Sepharose column (Amersham Biosciences AB, Uppsala, Sweden) equilibrated in 20 mM Tris pH 7.5 and proteins are eluted using a linear gradient from 0 to 300 mM NaCl. Appropriate fractions are pooled, acetonitrile is added to a final concentration of 10%, and the material is loaded onto a 10×250 mm, 10 micron, C4 RP-HPLC column (Vydac, Hesperia Calif., USA) equilibrated with 0.1 % TFA in water. Proteins are eluted using a linear gradient from 10 to 60% acetonitrile.

Relevant fractions are pooled and loaded onto a Superdex 200 26/60 column (Amersham Biosciences AB, Uppsala, Sweden) equilibrated in 1×PBS pH7. Appropriate fractions are pooled and concentrated. Final analysis to confirm the integrity of protein preparations utilizes MALDI mass analysis and N-terminal sequence analysis. Purified proteins are aliquoted and stored at −20 C for future use.

### Preparation 3

### Expression of an FGF-21 Compound in Yeast

Yet another expression system for production of an FGF-21 compound is yeast, such as Pichia pastoris, Pichia methanolica or Saccharomyces cerevisiae. For production in Pichia pastoris, a commercially available system (Invitrogen, Carlsbad, Calif.) uses vectors with the powerful AOX1 (alcohol oxidase) promters to drive high-level expression of recombinant proteins. Alternatively, vectors that use the promoter from the GAP gene (glyceraldehyde-3-phosphate dehydrogenase) are available for high level constitutive expression. The multi-copy Pichia expression vectors allow one to obtain strains with multiple copies of the gene of interest integrated into the genome. Increasing the number of copies of the gene of interest in a recombinant Pichia strain can increase protein expression levels.

### EXAMPLE 1

### Glucose Uptake in Mouse 3T3-L1 Adipocytes

3T3-L1 cells are obtained from the American Type Culture Collection (ATCC, Rockville, Md.). Cells are cultured in growth medium (GM) containing 10% iron-enriched fetal bovine serum in Dulbecco's modified Eagle's medium. For standard adipocyte differentiation, two days after cells reached confluency (referred as day 0), cells are exposed to differentiation medium (DM) containing 10% fetal bovine serum, 10 μg/ml of insulin, 1 μM dexamethasone, and 0.5 μM isobutylmethylxanthine, for 48 h. Cells then are maintained in post differentiation medium containing 10% fetal bovine serum, and 10 μg/ml of insulin.

Glucose Transport Assay—Hexose uptake, as assayed by the accumulation of 0.1 mM 2-deoxy-D-[¹⁴C]glucose, is measured as follows: 3T3-L1 adipocytes in 12-well plates are washed twice with KRP buffer (136 mM NaCl, 4.7 mM KCl, 10 mM NaPO₄, 0.9 mM CaCl₂, 0.9 mM MgSO₄, pH 7.4) warmed to 37° C. and containing 0.2% BSA, incubated in Leibovitz's L-15 medium containing 0.2% BSA for 2 h at 37° C. in room air, washed twice again with KRP containing, 0.2% BSA buffer, and incubated in KRP, 0.2% BSA buffer in the absence (Me₂SO only) or presence of wortmannin for 30 min at 37° C in room air. Insulin is then added to a final concentration of 100 nM for 15 min, and the uptake of 2-deoxy-D-[¹⁴C]glucose is measured for the last 4 min. Nonspecific uptake, measured in the presence of 10 μM cytochalasin B, is subtracted from all values. Protein concentrations are determined with the Pierce bicinchoninic acid assay. Uptake is measured routinely in triplicate or quadruplicate for each experiment.

In vitro potency (EC₅₀) is compared to the in vitro activity of wild-type FGF-21. The in vitro potency of PEGylated FGF-21 compounds of the present invention is compared to wild-type FGF-21 in Table 1. As indicated in Table 1, the PEGylated FGF-21 compounds of the present invention have reduced in vitro potency to various degrees compare to wild-type FGF-21. However, the decrease in in vitro potency is likely compensated for with the increase in time extension (plasma half life) of the PEGylated FGF-21 compounds.

TABLE 1

| | In vitro |
|---|---|
| | Potency |
| FGF-21 Compound | EC₅₀ (nM) |
| Wild-type | 0.57 |
| FGF-21 [K59C] | 0.85 |
| FGF-21 [K122C] | 0.82 |
| FGF-21 [K59C]-PEG* | 25.02 |
| FGF-21 [K122C]-PEG* | 21.87 |
| FGF-21 -PEG* | 191.1 |

*40 kDa polyethylene glycol-maleimide (PEG-maleimide)

### EXAMPLE 2

### 40kDa-PEG-Maleimide Reaction with FGF-21 Compounds

FGF-21 compounds such as K59C and K122C are selectively PEGylated at the introduced cysteine residue using maleimide-activated bifurcated 40 kDa mPEG (Nektar Therapeutics). For the PEGylation reaction, the peptide to be PEGylated is dissolved in 100 mM TRIS buffer at pH 8.0 and a 1.25-fold molar excess of bulk 40 kDa-mPEG is added. The reaction is allowed to stir at room temperature for 2-3 hours and then dialyzed overnight (7 kDa membrane) against 10 mM citrate, 10 mM phosphate, pH 7.4 at approximately 5° C. The PEGylated-FGF-21 compounds are purified by anion exchange chromatography on a Mono-Q column (Amersham Biosciences Corp, Piscataway, N.J.) using a NaCl gradient at neutral pH.

### EXAMPLE 3

### 20kDa-PEG-Maleimide Reaction with FGF-21 Compounds

FGF-21 compounds such as K59C, K122C, or K59C K122C are selectively PEGylated at the engineered cysteine residues using maleimide-activated linear 20 kDa mPEG (Nektar Therapeutics). For the PEGylation reaction, the peptide to be PEGylated is dissolved in 100 mM TRIS buffer at pH 8.0 and a 1.25-fold molar excess (per sulfhydryl) of bulk 40 kDa-mPEG is added. The reaction is allowed to stir at room temperature for 2-3 hours and then dialyzed overnight (7 kDa membrane) against 10 mM citrate, 10 mM phosphate, pH 7.4 at approximately 5° C. The PEGylated-FGF-21 compounds are purified by anion exchange chromatography on a Mono-Q column (Amersham Biosciences Corp, Piscataway, N.J.) using a NaCl gradient at neutral pH. For a doubly PEGylated molecule such as FGF21 K59C K122C, mono-PEGylated species are separated from double-PEGylated species by size exclusion chromatography on Superdex 200 (Amersham Biosciences Corp., Piscataway, N.J.) using a buffer of neutral pH.

### EXAMPLE 4

### Pharmacokinetic Analysis of PEGylated FGF-21 Compounds

PEGylated FGF-21 compound is administered by intravenous (IV) or subcutaneous (SC) routes at a dose of 0.4 mg/kg to CD-1 mice. The animals are bled at various times between 0 and 336 hours after dosing. Plasma was collected from each sample and analyzed by radioimmunoassay. Pharmacokinetic parameters are calculated using model-dependent (IV data) and independent (SC data) methods (WinNonlin Pro) and are reported in Table 2 below. By IV administration, the PEGylated FGF-21 compound has an elimination half-life of approximately 32.1 hours compared to an elimination half-life of 0.5 hours for native FGF-21. By SC administration the PEGylated FGF-21 compound has an elimination half-life of approximately 30.2 hours compared to an elimination half-life of 0.6 hours for native FGF-21. By both routes of administration the PEGylated FGF-21 compound demonstrates prolonged time action when compared to native FGF-2 1.

TABLE 2

| | | | | | | CL/F^{e} | |
|---|---|---|---|---|---|---|---|
| | | Cₘₐₓ^{a} | Tₘₐₓ^{b} | AUC_{0-∞}^{c} | t_{1/2}^{d} | (mL/h/ | % |
| Compound | Route | (ng/mL) | (d) | (ng*h/mL) | (h) | kg) | F^{f} |
| FGF-21-40 | IV | 6298 | — | 149534 | 32.1 | 2.7 | |
| kDa | | | | | | | |
| PEG | SC | 1641 | 12 | 88968 | 30.2 | 4.5 | 59 |
| FGF-21 | IV | 4300 | — | 1200 | 0.5 | 803 | — |
| | SC | 440 | 1.0 | 980 | 0.6 | 1024 | 78 |

^{a}Maximum observed plasma concentration.
^{b}Time of maximum observed plasma concentration.
^{c}Area under the plasma concentration-time curve measured from 0 to infinity.
^{d}Elimination half-life in hours.
^{e}Total body clearance as a function of bioavailability.
^{f}Percent bioavailability.

In another study, PEGylated FGF-21 compound or native FGF-21 are administered by bolus intravenous injection (IV) at a dose of 0.5 mg/kg to cynomolgus monkeys. The animals are bled at various times between 0 and 160 hours after dosing. Plasma was collected from each sample and analyzed by radioimmunoassay. Pharmacokinetic parameters are calculated using model-dependent (IV data) methods (WinNonlin Pro) and are reported in Table 3 below. PEGylated FGF-21 has an elimination half-life of approximately 75 hours while native FGF-21 has an elimination half-life of 2 hours, thus demonstrating the extended time action of the PEGylated FGF-21 compounds of the present invention. Furthermore, a skilled artisan recognizes that the extended time action of the PEGylated FGF-21 compounds is a result of the PEG moiety and is not dependent on the location of the PEG moiety on the FGF-21 compound. Thus, attaching the PEG moiety via a lysine residue or a cysteine residue will result in a PEGylated FGF-21 compound with extended time action characteristics allowing for fewer administrations of the PEGylated FGF-21 compound while maintaining a high blood level of the compound over a prolonged period of time.

TABLE 3

| | | AUC_{0-∞}^{a} | t_{1/2}^{b} | CL/F^{c} |
|---|---|---|---|---|
| Compound | Route | (μg*h/mL) | (d) | (mL/h/kg) |
| FGF-21-40 kDa | IV | 815 | 75 | 0.6 |
| PEG | | | | |
| FGF-21 | IV | 2.4 | 2.0 | 217 |

^{a}Area under the plasma concentration-time curve measured from 0 to infinity.
^{b}Elimination half-life in days.
^{c}Total body clearance as a function of bioavailability.

### EXAMPLE 5

### Ob/ob Mouse Model

The Ob/ob mouse model is an animal model for hyperglycemia, insulin resistance and obesity. Male ob/ob mice are used to monitor plasma glucose levels and triglyceride levels after treatment with PEGylated FGF-21 compounds compared to FGF-21 alone.

The test groups of male ob/ob mice (7 weeks old) are: (1) FGF-21, 5 μg/day for seven days; (2) FGF-21, 2.55 nM, administered on Day 0 only; (3) PEGylated FGF-21 2.55 nM;administered on Day 0 only; and (4) s.c. vehicle control (0.9% NaCl, 0.1 ml/mouse) for seven days. PEGylated FGF-21 and FGF-21 is administered s.c. in 0.1 ml.

The animals of groups (1) and (4) are dosed daily for 7 days and groups (2) and (3) are dosed on day 0 only. Blood glucose levels are measured daily for 10 days, 1 hour post dosing, using a standard protocol. The extended time action of PEGylated FGF-21 is indicated in Table 4 where a single dose on day 0 lowers blood glucose levels for 10 days.

TABLE 4

| | Blood Glucose Levels in ob/ob mice (mg/dl)* | | | | | | | |
|---|---|---|---|---|---|---|---|---|
| | Days of Treatment | | | | | | | |
| Treatment | 0 | 1 | 2 | 3 | 4 | 6 | 8 | 10 |
| Veh. Ctl. | 245 | 301 | 316 | 361 | 294 | 305 | 268 | 307 |
| (s.c.) | | | | | | | | |
| FGF-21 | 258 | 316 | 265 | 275 | 253 | 237 | 200 | 245 |
| 5 μg/day for | | | | | | | | |
| 7 days | | | | | | | | |
| FGF-21 | 249 | 287 | 268 | 308 | 275 | 271 | 270 | 288 |
| 2.55 nM, | | | | | | | | |
| day 0 only | | | | | | | | |
| PEGylated | 246 | 202 | 230 | 260 | 260 | 271 | 241 | 265 |
| FGF-21 | | | | | | | | |
| 2.55 nM, | | | | | | | | |
| day 0 only | | | | | | | | |

*Glucose levels measured 1 hour post dose

In another experiment, male ob/ob mice are used to monitor plasma glucose levels after a single treatment with PEGylated FGF-21 compounds compared to continuous infusion of FGF-21 alone. The test groups of male ob/ob mice (7 weeks old) are: (1) vehicle control (0.9% NaCl) by continuous infusion for seven days (Alzet pumps 1007D, 100 mcl, 0.5 mcl/h); (2) FGF-21, 3.4 nM by continuous infusion for seven days; (3) PEGylated FGF-21 3.4 nM; administered s.c. in 0.1 ml on Day 0 only; and (4) PEGylated FGF-21 compound K59C (cysteine PEGylation) 3.4 nM administered s.c. in 0.1 ml on Day 0 only; (5) PEGylated FGF-21 compound K122C (cysteine PEGylation) 3.4 nM administered s.c. in 0.1 ml on Day 0 only;

The animals of groups (1) and (2) are dosed by continuous infusion for 7 days and groups (3) and (5) are dosed on day 0 only. Blood glucose levels are measured daily for 7 days, 1 hour post dosing, using a standard protocol. The superior extended time action of PEGylated FGF-21 compound K122C is indicated in Table 5 where a single dose on day 0 lowers blood glucose levels for 7 days. PEGylated FGF-21 and PEGylated FGF-21 compound K59C also demonstrated blood glucose lowering effects as indicated in Table 5.

TABLE 5

| | Blood Glucose Levels in ob/ob mice (mg/dl)* | | | | | | | |
|---|---|---|---|---|---|---|---|---|
| | Days of Treatment | | | | | | | |
| Treatment | 0 | 1 | 2 | 3 | 4 | 5 | 6 | 7 |
| Veh. Ctl. | 260 | 249 | 296 | 248 | 294 | 292 | 277 | 270 |
| Continuous | | | | | | | | |
| infusion | | | | | | | | |
| FGF-21 | 261 | 183 | 189 | 124 | 153 | 139 | 124 | 122 |
| 3.4 nM | | | | | | | | |
| Continuous | | | | | | | | |
| infusion | | | | | | | | |
| PEGylated | 260 | 206 | 244 | 193 | 240 | 245 | 237 | 236 |
| FGF-21 | | | | | | | | |
| 3.4 nM, day | | | | | | | | |
| 0 only | | | | | | | | |
| PEGylated | 266 | 208 | 232 | 187 | 228 | 266 | 246 | 281 |
| FGF-21 K59C | | | | | | | | |
| 3.4 nM, day | | | | | | | | |
| 0 only | | | | | | | | |
| PEGylated | 256 | 176 | 222 | 198 | 179 | 205 | 198 | 204 |
| FGF-21 | | | | | | | | |
| K122C | | | | | | | | |
| 3.4 nM, day | | | | | | | | |
| 0 only | | | | | | | | |

*Glucose levels measured 1 hour post dose

### EXAMPLE 6

### Construction of DNA Encoding FGF-21 Compound K59C and K122C

pJB02 is an expression vector with an engineered leader peptide for efficient secretion of proteins in mammalian cell lines. Recombinant plasmid, pJB02/FGF21 (see P16820), where cDNA encoding wild type FGF-21 is inserted between AgeI and XbaI, respectively, and is used as a template to introduce site directed mutations to generate K59C and K122C variants of FGF-21 by means of SOE (Strand Overlapping Extension) PCR (Polymerase Chain Reaction). The typical conditions for PCR amplification are as follows: denaturation at 95° C. for 5 min, followed by 25 cycles of 1 min denaturation at 95° C., 1 min annealing at 55° C. and 1 to 2 min extension at 72° C., followed by a final extension at 72° C. for 7 minutes and cooling of the reaction at 4° C.

The following internal mutagenic primers (C+ and B−) are used for K59C:

Forward primer (5′, C+):
GTCTCCTGCAGCTGAAAGCCTTGTGCCCGGGAGTTATTCAAATCTTGGG
Reverse primer (3′, B−):
CCCAAGATTTGAATAACTCCCGGGCACAAGGCTTTCAGCTGCAGGAGAC

The following internal mutagenic primers (C+ and B−) are used for K122C:

Forward primer (5′, C+):
CGGCCTCCCGCTGCACCTGCCCGGGAACTGCTCCCCACACCGGGACCCTG
CAC
Reverse primer (3′, B−):
GTGCAGGGTCCCGGTGTGGGGAGCAGTTCCCGGGCAGGTGCAGCGGGAGG
CCG

The external amplification primers (A+ and D−) for both constructs are:

Forward primer (5′, A+):
GGACTTACCGGTCACCCCATCCCTGACTCCAGTCCTCTCCTGCAATTCGG
Reverse primer (3′, D−):
CTGTCTCTAGATCGAAGCTTTTATCAGGAAGCGTAGCTGGGGCTTCGGCC
CTGGGAAGGTCCCACCATGC

The SOE PCR is performed as follows:

Two PCRs are performed using pJB02/FGF21 as the template, with primers A+ and B− for one reaction and primers C+ and D− for the other. The PCRs result in two fragments: AB fragments of 212 and 400 bp (base pair) for K59C and K122C, respectively, and CD fragments of 418 and 234 bp for K59C and K122C, respectively. In the subsequent PCR, about equal molar amounts of AB and CD are added as the overlapping template and amplified with external primers, A+ and D A desired 581 bp PCR product, designated AD fragment, containing FGF-21 K59C or FGF-21 K122C is obtained. The final PCR product is subjected to digestion with restriction endonucleases, AgeI and XbaI, purified by preparative agarose gel electrophoresis and ligated to appropriately digested vector pJB02 fragment to generate a recombinant plasmid, pJB02/FGF-21 K59C or FGF-21K 122C. Both insert sequences are confirmed by DNA sequence analysis.

1. A PEGylated FGF-21 compound comprising an FGF-21 compound covalently attached to at least one PEG molecule, wherein each PEG is attached to the FGF-21 compound at a cysteine or lysine amino acid residue and wherein the PEGylated FGF-21 compound has extended time action compared to a non-PEGylated FGF-21 compound.

2. The PEGylated FGF-21 compound of claim 1 comprising the amino acid sequence as shown in SEQ ID NO:1 wherein said compound is selected from the group consisting of:
(a) a compound covalently attached to a PEG molecule at one or more lysine residues at positions 56, 59, 69 or 122: and,
(b) a compound covalently attached to a PEG molecule, at one or more amino acid residues selected from the group consisting of D25, D38, L58, K59, P60, K69, D79, H87, E91, E101, D102, L114, L116. K122, R126, P130, P133, or P140C, wherein said amino acid residue is substituted with a cysteine residue.

3. (canceled)

4. (canceled)

5. (canceled)

6. (canceled)

7. (canceled)

8. (canceled)

9. (canceled)

10. (canceled)

11. The PEGylated FGF-21 compound of claim 2 wherein said PEG molecule has a molecular weight of about 20,000 to 40,000 daltons.

12. A pharmaceutical composition useful for treating a patient exhibiting obesity, type 2 diabetes, insulin resistance, hyperinsulinemia, glucose intolerance, hyperglycemia, or metabolic syndrome comprising the following:
a. A therapeutically effective amount of the PEGylated FGF-21 compound of claim 1; and
b. An acceptable pharmaceutical carrier.

13. (canceled)

14. (canceled)

15. (canceled)

16. (canceled)

17. A method for treating a patient exhibiting obesity, type 2 diabetes, insulin resistance, hyperinsulinemia, glucose intolerance, hyperglycemia, or metabolic syndrome comprising administering to said patient in need of such treatment a therapeutically effective amount of the FGF-21 mutein of claim 1.

18. The method of claim 17 wherein said patient exhibits type 2 diabetes.

19. The method of claim 17 wherein said patient exhibits obesity.

20. The method of claim 17 wherein said patient exhibits metabolic syndrome.

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)
